(19) United States
(12) Patent Application Publication
(10) Pub. No.: US 2011/0269841 A1
(43) Pub. Date: Nov. 3, 2011

(54) THIN FILM WITH METHADONE ACTIVE INGREDIENT

(75) Inventor: John LeDonne, Ridgewood, NJ (US)

(73) Assignee: INNOTEQ, INC., Stratford, CT (US)

(21) Appl. No.: 13/100,232

(22) Filed: May 3, 2011

Related U.S. Application Data
(60) Provisional application No. 61/330,486, filed on May 3, 2010.

(51) Int. Cl. A61K 31/137; B29D 7/01

(52) U.S. Cl. 514/648; 264/212

(57) ABSTRACT

An orally dissolvable thin film strip for the administration of the pharmaceutical active Methadone is formed by mixing a wet slurry of a water-soluble polymer, a polar solvent, Methadone HCl, and optional additives. The slurry is then cast onto a substrate by slot die or knife-over-roll and is dried in an oven at a temperature that exceeds the boiling point of the solvent. The resulting dried thin film is cut into individual doses designed to administer 5 mg, 10 mg, or 40 mg of Methadone HCl.

### CROSS-REFERENCE TO RELATED APPLICATIONS

This application claims priority to, and the benefit of, U.S. Provsional Application Ser. No. 61/330,486 filed on May 3, 2010, the disclosure of which is incorporated herein by reference in its entirety.

### FIELD

The aspects of the disclosed embodiments relate to rapidly dissolving films and methods of their preparation and administration to a patient. More specifically, they relate to rapidly dissolving films for the oral administration of Methadone.

### BACKGROUND

Active ingredients, such as drugs or pharmaceuticals, may be prepared in a pill, capsule or tablet form to allow for accurate and consistent dosing. However, this form of preparing and dispensing medications has many disadvantages including that a large proportion of adjuvants that must be added to obtain a size able to be handled, that a larger medication form requires additional storage space, and that dispensing includes counting the tablets which has a tendency for inaccuracy.

Many people have difficulty swallowing pills, capsules and tablets. While some of these dosage forms may be cut or split to facilitate administration tablets this is not a suitable solution for many forms and, for example, may destroy the controlled release properties.

Thus there exists a need for an alternative to solid dosage forms.

### SUMMARY

One aspect of the disclosed embodiments relates to a method of forming a thin film strip. The method includes mixing a liquid solvent component, a water-soluble polymer component, and a Methadone active ingredient to form a matrix; depositing the matrix onto a substrate; drying the matrix in a drying oven; wherein the temperature of the matrix is substantially greater than the boiling point of the solvent while drying in the drying oven.

Another aspect of the disclosed embodiments relates to a thin film strip having a Methadone active ingredient. The orally dissolvable thin film is composed of 5-50% weight Methadone HCl; 1-50% weight water-soluble polymer; 1-15% plasticizer; and 0-20% weight polar solvent.

### DETAILED DESCRIPTION OF THE DISCLOSED EMBODIMENTS

This disclosure describes the best mode or modes of practicing the invention as presently contemplated. This description is not intended to be understood in a limiting sense, but provides an example of the invention presented solely for illustrative purposes by reference to the accompanying drawings to advise one of ordinary skill in the art of the advantages and construction of the invention.

The disclosed thin film is generally a thin, dissolvable film. In one embodiment, the formed film is adapted to be an edible film for oral intake and use for the purpose of dispensing medicaments or active ingredients. In alternate embodiments, the thin strip may be adapted to transdermally deliver the active ingredient via application to the skin surface of a body part.

In one embodiment, the thin film product is formed from a matrix composed of a solvent or liquid, a water-soluble polymer and a pharmaceutically active ingredient incorporated within the polymer matrix.

In one embodiment, the solvent is water. In alternate embodiments, other polar solvents may be used.

Where the film forming polymer is water soluble it may be composed of, but is not limited to polymers selected from pullulan, hydroxypropylmethyl cellulose, hydroxyethyl cellulose, hydroxypropyl cellulose, polyvinyl pyrrolidone, carboxymethyl cellulose, polyvinyl alcohol, sodium alginate, polyethylene glycol, pectin, tragacanth gum, guar gum, acacia gum, Arabic gum, polyacrylic acid, methylmethacrylate copolymer, carboxyvinyl polymer, gelatin, amylase, high amylase starch, hydroxypropylated high amylase starch, dextrin, chitin, chitosan, levan, elsinan, collagen, zein, gluten, soy protein isolate, whey protein isolate, casein and mixtures thereof.

Recognized film-forming agents include xantha gum, polysaccharides, natural gums, polypeptides, polyacrylates, starch, karaya gum, mixtures thereof and others.

Other film forming agents which can be used include, but are not limited to, cellulose ethers, modified starches, natural gums; edible polymers, hydrocolloid flours, seaweed extracts, land plant extracts, derivatives thereof, and combinations thereof.

In one embodiment, the water soluble polymer of the film forming matrix of the disclosed embodiments is one of pullulan, hydroxypropylethyl cellulose, hydroxyethyl cellulose, hydroxypropyl cellulose, carboxymethyl cellulose, sodium alginate, pectin, and mixtures thereof. It should be appreciated by those skilled in the art that there are other edible water-soluble film forming agents that exhibit desirable properties and may be used.

In general, the effective amount of the film forming agent ranges from approximately about 10% to about 90%, preferably about 25% to about 75% dry weight of the film composition.

Other ingredients or additives may be added to the blend for the various purposes and include, but are not limited to, bulk fillers, binding agents, thickening agents, softeners or plasticizing agents, surfactants, stabilizing agents, emulsifiers, buffers, disintegrants and other additives or components such as, for example, artificial or natural flavors, artificial or natural sweeteners, colorants, and the like, which may provide a benefit to the user but which are not considered essential to the forming of the matrix nor pharmaceutically active.

In one embodiment, the pharmaceutically active ingredient of the thin film strip is Methadone. In one embodiment, the Methadone active is in the form of Methadone HCl. In alternate embodiments, the Methadone active may be any pharmaceutically active Methadone salt. Methadone is useful in the treatment of opioid dependence. It has cross-tolerance with other opioids including heroin and morphine and a long duration of effect: oral doses of methadone can stabilize patients by mitigating opioid withdrawal syndrome. Higher doses of methadone can block the euphoric effects of heroin, morphine, and similar drugs. As a result, properly dosed methadone patients can reduce or stop altogether their use of these substances.

The advantages of a Methadone strip are the inability to “cheek” the dosage form as it will dissolve in less than 10 seconds in the buccal cavity. There may also be less potential for gastrointestinal distress.

In one embodiment, the thin film strip version of Methadone is designed to orally administer a single dose of Methadone. In one embodiment, the dose amount of Methadone in each strip may be 5, 10 or 40 mg. The Methadone strips may be individually packaged, for ease of administration, and to lessen confusion of dosing.

In one embodiment, the Methadone active may be the sole pharmaceutically active ingredient included in the thin film strip. In an alternate embodiment, the Methadone may additionally be encapsulated or may be combined with other active ingredients for increased therapeutic benefits.

In one embodiment, the composition of the thin film strip is designed to hold a large amount of the pharmaceutical active Methadone and to include minimal amounts of film formers, plasticizers, and other components. The reason for this is to ensure the fastest dissolution time. Film formers add film strength and elasticity, but they also increase dissolution time.

In an alternate embodiment, the film strip may include larger amounts of plasticizers or film formers to create a more flexible film. There are limitations to the amount of the plasticizer that can be used in the film. When excessive plasticizer is employed, the film loses structural integrity, becomes too flimsy and, significantly, becomes sticky such that it tends to adhere to other strips of film in the package forming a block. This is especially true when the strips are stacked in a vial, which is a preferred form of primary packaging.

In one embodiment, the method of forming the thin strip includes coating a liner substrate or stainless steel belt with a wet slurry of the film forming ingredients, including the liquid or solvent, the water soluble polymer, the pharmaceutical active, and any other additives or components. The wet slurry is then dried in a drying oven to remove at least a portion of the solvent from the slurry to form a film.

In one embodiment, the process for manufacturing the Methadone-containing thin film strip can include forming the wet slurry or matrix by mixing all of the film-forming ingredients in a blending tank to ensure homogeneous slurry. The pharmaceutically active Methadone is added toward the end of the blending process and is paddle mixed to ensure uniform distribution without crushing any possible encapsulations. The matrix is then moved into a holding tank where it is kept agitated to ensure continued homogeneity.

The matrix is then deposited or coated, either though a slot die or under a knife-over-roll, onto a release liner substrate, which is then passed into an oven. The drying oven is generally adapted to dry the slurry by removing most or all of the liquid solvent while, at the same time, not heating the pharmaceutical active above its degradation temperature, in order to maintain efficacy of the medicament or active. In the oven, in one embodiment, the matrix is exposed to a temperature that is in the range of approximately 230° F. to 240° F. for a period of approximately 7 to 15 minutes. Test results show that the temperature of the matrix while in the oven reaches approximately 227° F. In one embodiment between approximately 80% to approximately 100% of the solvent is removed during the drying process.

In one embodiment, after the dried film is removed from the oven, the film is cured for a desired period of time. The curing process may be carried out in a foil bag to reduce moisture loss or acquisition, or avoid the flashing of flavors or other volatile additives, if used. The curing process typically lasts 2 to 5 days, most typically approximately 4 days.

In one embodiment, the substrate can be delaminated from the dried film. In an alternate embodiment, the substrate can be delaminated from the coated slurry after the coating stage.

The dried film may be cut into single dose sizes and various shapes using conventional means for subsequent packaging. The individual, cut films are sized in order to contain 5 mg, 10 mg, or 40 mg of Methadone HCl active for administration. The cut thin films, according to one embodiment, are of a size and shape suitable to be orally consumed by a human or animal. Where the thin strip is designed for topical application of the active ingredient, the strip may be cut into other shapes suitable for application to a body part.

The finished film is preferably packaged in moisture retardant packaging.

In one embodiment, the finished thin film product is a single-layer film that is between 0.1 and 20 mil thick based on dose. In alternate embodiments, a multi-layer film may be formed by laminating multiple layers of thin films to one another, where the films have physical and/or chemical properties which are modified depending on the function each layer plays in the laminate structure. Multiple layers may increase the amount of active that may be used, or may facilitate the addition of plasticizers or other additives or facilitate the incorporation of more than one active ingredient, where the actives would react negatively with one another. In general, there may be any number of layers of film. In one embodiment, 3 to 5 layers may be used. In one embodiment, the layers may be manufactured or formed individually and combined after manufacture. However, in an alternate embodiment, the thin films of each layer are manufactured simultaneously.

In one embodiment, the resulting thin film may display minimal or no viscoelastic properties because of the large amount of active with minimal plasticizers or film formers.

In one embodiment, the composition of the resulting dried film is 5-50% polymer, 1-50% pharmaceutically active ingredient, 1-15% plasticizer, 0-20% solvent, 0-25% flavor, 0-10% surfactant, 0-20% emulsifier, 0-20% thickening agent, 0-20% disintegrant, 0-25% sweetener, 0-20% buffer.

One non-limiting example of a thin film strip prepared in accordance with aspects of the disclosed embodiments has the composition of 5-50% Hypromellose, 1-50% Methadone HCl, 1-15% Glycerin, 0-20% Water, 0-25% Flavor, 0-10% tween 80, 0-20% gum arabic, 0-20% xanthan gum, 0-20% maltodextrin, 0-25% sucralose or other artificial sweetener, 0-20% phosphate.

It is noted that all ranges included in the above disclosure are inclusive of end points.

The aspects of the disclosed embodiments relate to a thin strip containing a Methadone active ingredient and a method of making the strip that includes mixing a wet slurry or matrix of film forming components, depositing the matrix onto a liner or substrate, drying the matrix in a drying oven at temperatures that exceed the boiling point of the solvent and are less than the degradation temperature of the active ingredient. The dried films can then be delaminated from the substrate and cut into smaller strips for individual doses of the active ingredient.

While the present invention has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the invention. Furthermore, the foregoing describes the invention in terms of embodiments foreseen by the inventor for which an enabling description was available, notwithstanding that insubstantial modifications of the invention, not presently foreseen, may nonetheless represent equivalents thereto.

What is claimed is:

1. A method of forming a thin film product comprising:
mixing a liquid solvent component, a water-soluble polymer component, and a Methadone active ingredient to form a matrix;
depositing the matrix onto a substrate;
drying the matrix in a drying oven;
wherein the temperature of the matrix is substantially greater than the boiling point of the solvent while drying in the drying oven.

2. The method according to claim 1 wherein the liquid solvent is water.

3. The method according to claim 1 wherein the matrix is deposited onto the liner substrate through a slot die.

4. The method according to claim 1 wherein the matrix is deposited onto the liner substrate by a knife-over-roll.

5. The method according to claim 1 wherein the oven temperature is in the range of approximately 110° C. to approximately 116° C.

6. The method according to claim 1 wherein the matrix is dried in the drying oven for a period of approximately 7 to approximately 15 minutes.

7. The method according to claim 1 wherein the temperature of the matrix reaches approximately 108° C. while in the drying oven.

8. The method according to claim 1 further comprising delaminating the film from the liner substrate.

9. The method according to claim 1 further comprising cutting the dried thin film product into individual doses for packaging.

10. The method according to claim 9, wherein the individual doses are 5 mg, 10 mg, or 40 mg of Methadone HCl.

11. The method according to claim 1 wherein the resulting thin film has a thickness of about 0.1 to about 20 mil.

12. An orally dissolvable thin film for the administration of Methadone comprising:
5-50% weight Methadone HCl;
1-50% weight water-soluble polymer;
1-15% plasticizer; and
0-20% weight polar solvent.

13. The thin film according to claim 12 wherein the water-soluble polymer is Hypromellose.

14. The thin film according to claim 12 wherein the plasticizer is glycerin

15. The thin film according to claim 12 wherein the polar solvent is water.
